(19) United States
(12) Patent Application Publication
(10) Pub. No.: US 2012/0065520 A1
(43) Pub. Date: Mar. 15, 2012

(54) OPTICAL MEASUREMENT APPARATUS

(75) Inventors: Hidetoshi IWASAKI, Kawasaki (JP); Takayuki Yamaji, Kawasaki (JP); Hiroki Itagaki, Kawasaki (JP)

(73) Assignee: FUJITSU LIMITED, Kawasaki (JP)

(21) Appl. No.: 13/185,049

(22) Filed: Jul. 18, 2011

(30) Foreign Application Priority Data
Sep. 15, 2010 (JP) 2010-206891

(51) Int. Cl. A61B 6/00

(52) U.S. Cl. 600/476

(57) ABSTRACT

An optical measurement apparatus has a light source section, a light receiving section, and a light guiding section. The light source section has a light emitting surface that emits light. The light receiving section has a light receiving surface that receives the light emitted by the emitting surface of the light source to be incident into a body through a surface of the body and which is emitted out of the body after propagating inside the body, the receiving surface facing a direction orthogonal to the emitting surface of the light source, and the receiving section outputting a signal indicative of an amount of the light received by the receiving surface. The light guiding section is provided on a path of the light emitted from the emitting surface and received by the receiving surface to change a traveling direction of the light.

### CROSS-REFERENCE TO RELATED APPLICATIONS

This application is related to and claims priority to Japanese Patent Application No. 2010-206891 filed on Sep. 15, 2010 and herein incorporated by reference.

### BACKGROUND

1. Field

The present invention relates to an optical measurement apparatus.

2. Description of the Related Art

An optical information measurement apparatus or the like including a light source section that illuminates a living body, a light receiving section that receives the light emitted from the light source section and emitted out of the living body through a surface of the living body after propagating inside the living body, a shaping section that shapes the surface of the living body, and a computation section that calculates information on the living body on the basis of the amount of the light received by the light receiving section is known in the art.

Japanese Laid-open Patent Publication No. 2000-237195, International Publication No. WO 1989/008428, Japanese Laid-open Patent Publication No. 2003-310575, Japanese Laid-open Patent Publication No. 2003-210465, International Publication No. WO 2004/110273, Japanese Laid-open Patent Publication No. 08-15142, Japanese Laid-open Patent Publication No. 2009-233285, Japanese Laid-open Patent Publication No. 2005-259361, and Japanese Laid-open Patent Publication No. 2006-184890 are related to such an optical information measurement apparatus.

### SUMMARY

It is an aspect of the embodiments discussed herein to provide an optical measurement apparatus which has: a light source section, a light receiving section, and a light guiding section. The light source section has a light emitting surface that emits light to be incident into a body. The light receiving section has a light receiving surface that receives the light which is emitted by the light emitting surface of the light source section to be incident into the body through a surface of the body and which is emitted out of the body after propagating inside the body, the light receiving surface facing a direction orthogonal to the light emitting surface of the light source section, and the light receiving section outputting a signal having a value corresponding to an amount of the light received by the light receiving surface. The light guiding section is provided on a path of the light emitted from the light emitting surface of the light source section and received by the light receiving surface of the light receiving section to change a traveling direction of the light.

### BRIEF DESCRIPTION OF THE DRAWING

FIG. 1 is a side view illustrating the arrangement relationship between a light source section (LED) and a light receiving section (PD) in a sensor section of an optical measurement apparatus according to a reference example;

FIG. 2 is a side view illustrating the arrangement relationship between a light source section (LED) and a light receiving section (PD) in a sensor section of an optical measurement apparatus according to a first embodiment of the present invention;

FIG. 3 is a side view illustrating an example of the arrangement of the light source section, the light receiving section, and a light guiding member in the sensor section of the optical measurement apparatus according to the first embodiment of the present invention;

FIG. 4A is a side view illustrating an example of the arrangement of a light source section, a light receiving section, and a light guiding member in a sensor section of an optical measurement apparatus according to a second embodiment of the present invention;

FIG. 4B is a side view illustrating the arrangement relationship between the light source section (LED) and the light receiving section (PD) in the sensor section of the optical measurement apparatus according to the second embodiment of the present invention (in which the arrangement relationship between the light source section and the light receiving section is reversed from that in FIG. 4A);

FIG. 4C is a bottom view illustrating an example of the arrangement of the light source section, the light receiving section, and the light guiding member in the sensor section of the optical measurement apparatus according to the second embodiment of the present invention (in which the arrangement relationship between the light source section and the light receiving section is reversed from that in FIG. 4A);

FIG. 4D is a side view illustrating the function of the light guiding member of the sensor section of the optical measurement apparatus according to the second embodiment of the present invention (in which the arrangement relationship between the light source section and the light receiving section is reversed from that in FIG. 4A);

FIG. 5 is a side view illustrating an example of the arrangement of a light source section, a light receiving section, and a light guiding member in a sensor section of an optical measurement apparatus according to a third embodiment of the present invention;

FIG. 6 is a side view illustrating an example of the arrangement of a light source section, a light receiving section, and a light guiding member in a sensor section of an optical measurement apparatus according to a fourth embodiment of the present invention;

FIG. 7 is a side view illustrating an example of the arrangement of a light source section, a light receiving section, and a light guiding member in a sensor section of an optical measurement apparatus according to a fifth embodiment of the present invention;

FIG. 8A is a side view illustrating the function of the sensor section of the optical measurement apparatus according to the second embodiment of the present invention;

FIG. 8B is a side view illustrating an example of the arrangement and the function of a light source section, a light receiving section, and a light guiding member in a sensor section of an optical measurement apparatus according to a sixth embodiment of the present invention;

FIG. 9 illustrates the function of the sensor section of the optical measurement apparatus according to each of the embodiments of the present invention;

FIG. 10A is a block diagram illustrating an example of the overall configuration of the optical measurement apparatus according to each of the embodiments of the present invention; and

FIG. 10B illustrates the function of a subcutaneous fat thickness calculation section illustrated in FIG. 10A.

### DETAILED DESCRIPTION OF THE EMBODIMENTS

The technology disclosed below provides an optical measurement apparatus that can be reduced in thickness.

The optical measurement apparatus disclosed below includes a light source section having a light emitting surface that emits light to be incident into a living body through a surface of the living body. The optical measurement apparatus further includes a light receiving section having a light receiving surface that receives the light which is emitted by the light emitting surface of the light source section to be incident into the living body through the surface of the living body and which is emitted out of the living body through the surface of the living body after propagating inside the living body. The light receiving surface of the light receiving section faces a direction along a plane orthogonal to the light emitting surface of the light source section. The light receiving section outputs a signal having a value corresponding to the amount of the light received by the light receiving surface. The optical measurement apparatus further includes a light guiding member that changes the traveling direction of the light emitted by the light emitting surface of the light source section such that the light is incident into the living body through the surface of the living body. Alternatively, the light guiding member changes the traveling direction of the light, which is emitted by the light emitting surface of the light source section to be incident into the living body through the surface of the living body and which is emitted out of the living body through the surface of the living body after propagating inside the living body, to guide the light to the light receiving surface of the light receiving section. The light which propagates inside the living body is hereinafter referred to as “propagation light”.

With the light receiving surface of the light receiving section facing a direction orthogonal to the light emitting surface of the light source section, the light emitting surface of the light source section or the light receiving surface of the light receiving section can be disposed to face the direction of extension of the surface of the living body. As a result, the optical measurement apparatus can be reduced in thickness in the case where the light source section is longer in the direction orthogonal to the light emitting surface than in the direction in parallel with the light emitting surface or in the case where the light receiving section is longer in the direction orthogonal to the light receiving surface than in the direction in parallel with the light receiving surface.

The optical measurement apparatus according to each of embodiments of the present invention described below is an apparatus that measures information (for example, the thickness of subcutaneous fat) on a living body (for example, the abdomen, an arm, a leg, or the like included in a human body). That is, an example of the optical measurement apparatus according to each of the embodiments of the present invention is a fat thickness measurement apparatus that calculates a fat thickness using light. The optical measurement apparatus obtains information on the living body on the basis of the amount of light that returns from the living body after being emitted to the living body and scattered in a fat layer, for example, in the living body or reduced through absorption by a surface of a muscle layer. The light which returns to the living body after being scattered in the fat layer, for example, in the living body or reduced through absorption by the surface of the muscle layer is occasionally referred to as “return light”.

The information on the living body to be measured may be the amount of a particular substance (for example, the amount of water) contained in the living body, rather than the thickness of subcutaneous fat. The amount of light at a particular wavelength absorbed varies between substances in the living body. Also, the amount of light absorbed by a particular substance varies depending on the wavelength of the light used. For example, visible light at a wavelength of 650 nm or less is well absorbed by hemoglobins, and light at a wavelength of 1100 nm or more is well absorbed by water. In addition, melanin stably absorbs light over a wide range of wavelengths. That is, substances in the living body are different in light absorption properties (amount by which to attenuate the propagation light) for various wavelengths. By utilizing these characteristics, a plurality of different wavelengths of light are incident into the living body and the amount of the return light from the living body is measured for each of the wavelengths of the incident light to analyze the measurement results. The amount of a particular substance in the living body can be measured in this way.

In the case where the optical measurement apparatus according to each of the embodiments of the present invention is used to measure the thickness of subcutaneous fat, light at a wavelength of 810 nm, for example, may be used. As described above, visible light at a wavelength of 650 nm or less is well absorbed by hemoglobins, and light at a wavelength of 1100 nm or more is well absorbed by water. In contrast, light at a wavelength of 650 to 1100 nm, which is between the above ranges is transmitted in a living body tissue with a high transmittance so as to be able to reach to the depths of the living body tissue without damaging the living body tissue. Thus, such light is referred to as a “window of light”. It is desirable to avoid light at a wavelength of 975 nm or more because light at a wavelength of 975 nm is well absorbed by water. Thus, as described above, in the case where the optical measurement apparatus according to each of the embodiments of the present invention is used to measure the thickness of subcutaneous fat, light at a wavelength of 810 nm, for example, may be used. Light at a wavelength of about 700 to 1500 nm, which includes 810 nm, is occasionally referred to as “near infrared light”.

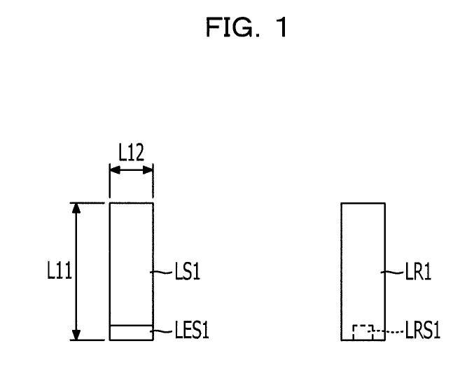

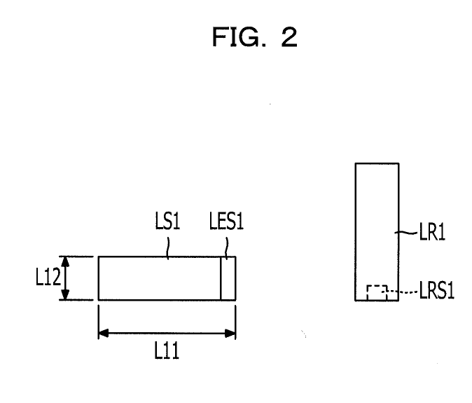

FIGS. 1 and 2 serve to illustrate the arrangement relationship between a light source section and a light receiving section in an optical measurement apparatus according to a first embodiment. The light source section is a device that emits light to be incident into the living body, and may be an LED (Light Emitting Diode), for example. The light receiving section is a device that receives the light, which is emitted by the light source section to be incident into the living body and which is emitted from the living body after propagating inside the living body, to output a signal corresponding to the amount of the received light, and may be a PD (Photo Diode), for example.

FIG. 1 illustrates the arrangement relationship between a light source section and a light receiving section in an optical measurement apparatus according to a reference example. In the example of FIG. 1, a light source section LS1 and a light receiving section LR1 are disposed with a light emitting surface LES1 and a light receiving surface LRS1, respectively, facing downward in the drawing. In the example of FIG. 1, the optical measurement apparatus is used with the light emitting surface LES1 of the light source section LS1 and the light receiving surface LRS1 of the light receiving section LR1 in contact with a surface of a living body to be measured (not illustrated). That is, in the example of FIG. 1, light emitted from the light emitting surface LES1 of the light source section LS1 is incident into the living body through the surface of the living body. A portion of the incident light that is scattered by the fat layer, for example, in the living body or reflected by the muscle layer to be emitted out of the living body through the surface of the living body, that is, return light, is received by the light receiving surface LRS1 of the light receiving section LR1. The length of the light source section LS1 in the direction orthogonal to the light emitting surface LES1 is L11, and the length of the light source section LS1 in the direction in parallel with the light emitting surface LES1 is L12, with L11>L12.

FIG. 2 illustrates the arrangement relationship between a light source section and a light receiving section in an optical measurement apparatus according to a first embodiment of the present invention. In the first embodiment of FIG. 2, a light source section LS1 is disposed with a light emitting surface LES1 facing rightward in the drawing, and a light receiving section LR1 is disposed with a light receiving surface LRS1 facing downward in the drawing. That is, in the first embodiment of FIG. 2, the light source section LS1 and the light receiving section LR1 are disposed such that the light emitting surface LES1 of the light source section LS1 is orthogonal to the light receiving surface LRS1 of the light receiving section LR1. The optical measurement apparatus according to the first embodiment of FIG. 2 is used with the light emitting surface LES1 of the light source section LS1 facing the direction of extension of a surface of a living body to be measured (not illustrated) and with the light receiving surface LRS1 of the light receiving section LR1 in contact with the surface of the living body. That is, in the first embodiment of FIG. 2, light emitted from the light emitting surface LES1 of the light source section LS1 is incident into the living body after a light guiding member to be discussed later changes the traveling direction of the light such that the light travels downward in the drawing. A portion of the incident light that is scattered by the fat layer, for example, in the living body or reduced through absorption by the muscle layer, that is, return light, is received by the light receiving surface LRS1 of the light receiving section LR1. Again, the length of the light source section LS1 in the direction orthogonal to the light emitting surface LES1 is L11, and the length of the light source section LS1 in the direction in parallel with the light emitting surface LES1 is L12, with L11>L12.

According to the optical measurement apparatus of the first embodiment of FIG. 2, as described above, the light source section LS1 and the light receiving section LR1 are disposed such that the light emitting surface LES1 of the light source section LS1 is orthogonal to the light receiving surface LRS1 of the light receiving section LR1. As described above, the length of the light source section LS1 in the direction orthogonal to the light emitting surface LES1 is L11, and the length of the light source section LS1 in the direction in parallel with the light emitting surface LES1 is L12, with L11>L12. While the height of the light source section LS1 is L11 in the arrangement of the reference example of FIG. 1, the height of the light source section LS1 is L12 in the arrangement of the first embodiment of FIG. 2. With L11>L12 as described above, the height of the light source section LS1 in the arrangement of the first embodiment of FIG. 2 is smaller than the height of the light source section LS1, which is L11, in the arrangement of the reference example of FIG. 1, and thus the optical measurement apparatus can be reduced in height at a portion corresponding to the light source section LS1.

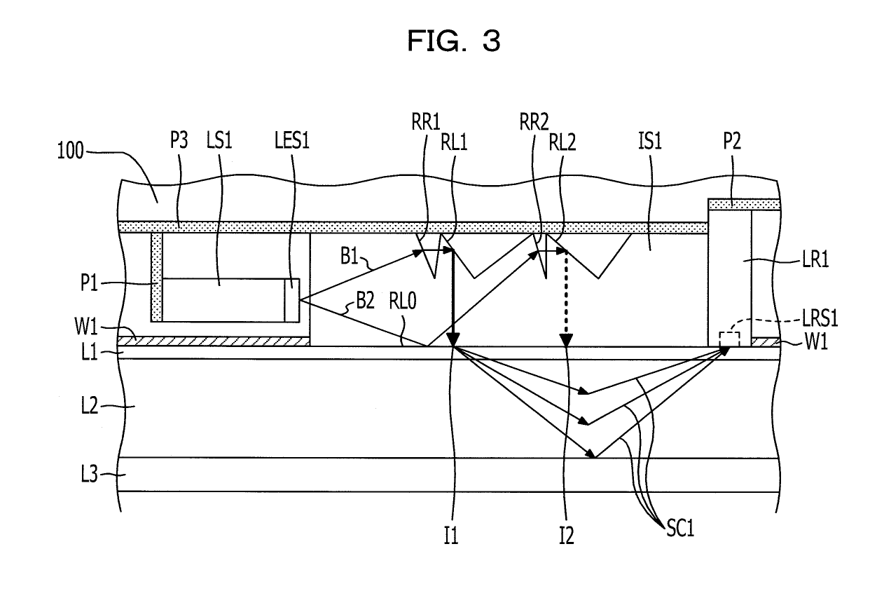

FIG. 3 is a side view illustrating an example of the arrangement of the light source section, the light receiving section, and a light guiding member in a sensor section of the optical measurement apparatus according to the first embodiment described above.

In FIG. 3, the light source section LS1 in the sensor section of the optical measurement apparatus according to the first embodiment is an LED as described above, and emits light at a wavelength of 810 nm, for example, as described above. The light receiving section LR1 in the sensor section is a PD as described above, and receives light at a wavelength of 810 nm to output a voltage (output signal) corresponding to the amount of the received light.

A light guiding member IS1 in the sensor section is made of a material that can transmit the light generated (emitted) by the light emitting surface LES1 of the light source section LS1. The material may be polycarbonate, for example. The light guiding member IS1 has a function of changing the traveling direction of the light generated (emitted) by the light emitting surface LES1 of the light source section LS1 through refraction and/or reflection so as to emit the light downward in FIG. 3.

The light source section LS1, the light receiving section LR1, and the light guiding member IS1 in the sensor section of the optical measurement apparatus illustrated in FIG. 3 are placed on the surface of the living body to be used. As illustrated in FIG. 3, the living body includes epidermis and dermis L1, a fat layer L2, and a muscle layer L3. In FIG. 3, an upper surface of the epidermis and dermis L1 serves as the surface of the living body.

In FIG. 3, the light emitted downward from the light guiding member IS1 as described above is incident into the living body through the surface of the living body. The light incident into the living body transmits through the epidermis and dermis L1 to reach the fat layer L2, and is scattered in the fat layer L2 to be absorbed by the muscle layer L3, or is reflected by the muscle layer L3. Part of the light scattered in the fat layer L2 or reflected by the muscle layer L3, that is, light SC1, is incident on the light receiving surface LRS1 of the light receiving section LR1 to be received.

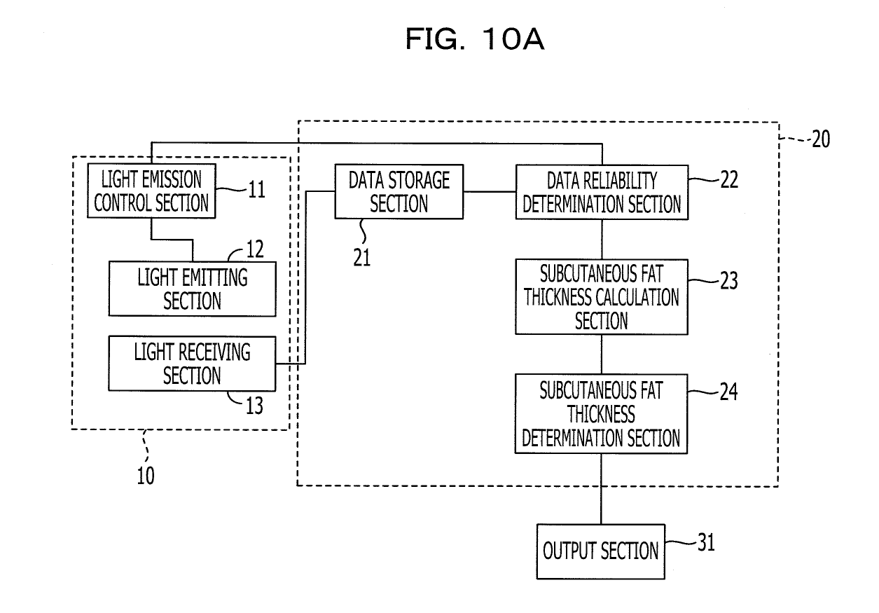

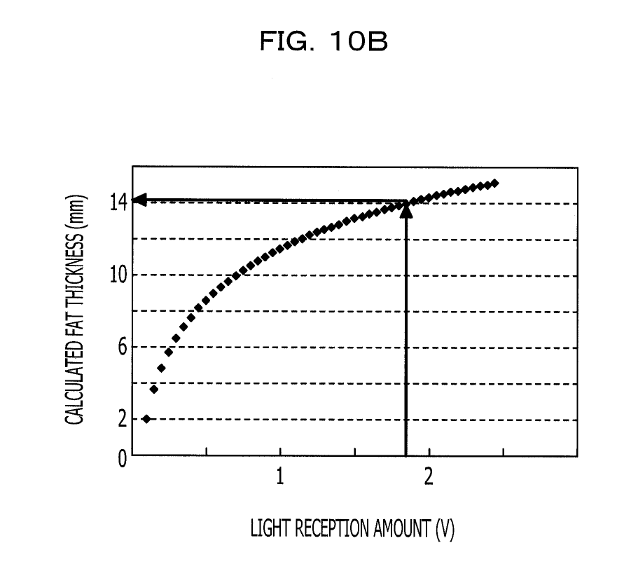

If the fat layer L2 is thin, a higher proportion of the light incident into the living body is absorbed by the muscle layer L3. As a result, the propagation light is attenuated by a larger amount, which reduces the amount of the light received by the light receiving surface LRS1 of the light receiving section LR1. If the fat layer L2 is thick, on the contrary, a lower proportion of the light incident into the living body is absorbed by the muscle layer L3. As a result, the propagation light is attenuated by a smaller amount, which increases the amount of the light received by the light receiving surface LRS1 of the light receiving section LR1. Thus, the amount of the light received by the light receiving surface LRS1 of the light receiving section LR1 is larger as the thickness of the fat layer L2 is larger (see FIG. 10B to be discussed later), and the thickness of the fat layer L2 of the living body can be measured by utilizing these characteristics.

In FIG. 3, light B1, which is part of the light incident into the light guiding member IS1 from the light emitting surface LES1 of the light source section 1, travels obliquely rightward and upward when the light B1 is incident into the light guiding member IS1 as illustrated in the drawing. After being incident into the light guiding member IS1, the light B1 is refracted by a refractive surface RR1 provided in the light guiding member IS1 so as to travel substantially horizontally rightward, then reflected by a reflective surface RL1 provided in the light guiding member IS1 so as to travel downward in FIG. 3, and incident into the living body (L1→L2) through an emission position I1.

Likewise, light B2, which is part of the light incident into the light guiding member IS1 from the light emitting surface LES1 of the light source section 1, travels obliquely rightward and downward when the light B2 is incident into the light guiding member IS1 as illustrated in the drawing. After being incident into the light guiding member IS1, the light B2 is reflected by a reflective surface RL0 provided in the light guiding member IS1 so as to travel obliquely rightward and upward. After that, the light B2 is refracted by a refractive surface RR2 provided in the light guiding member IS1 so as to travel substantially horizontally rightward. The light B1 is then reflected by a reflective surface RL2 provided in the light guiding member IS1 so as to travel downward in FIG. 3, and incident into the living body (L1→L2) through an emission position 12.

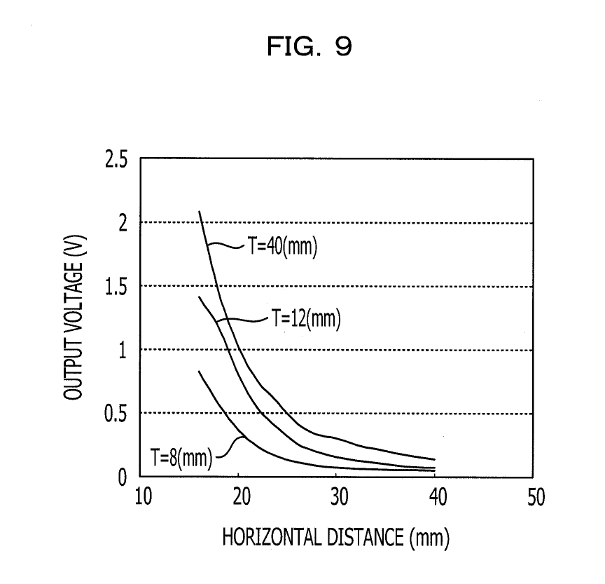

With the light B1 and the light B2 incident into the living body (L1→L2) through the two emission positions 11 and 12, respectively, in this way, a larger amount of light is incident into the living body (L1→L2) compared to a case where light is incident into the living body (L1→L2) only through a single emission position. As a result, a larger amount of light reaches the light receiving surface LRS1 of the light receiving section LR1 to be received after propagating inside the living body (L1, L2), which increases the difference in voltage of the output signal from the light receiving section LR1 for a diversity of the thickness of the fat layer L2. Hence, the sensitivity of the optical measurement apparatus according to the first embodiment in measuring the thickness of the fat layer L2 of the living body is improved (which will be discussed later with reference to FIG. 9). Each of the emission positions 11 and 12 can be deemed as an artificial light source. In FIG. 3, two artificial light sources are provided in correspondence with the two emission positions 11 and 12.

Methods of forming the refractive surfaces RR1 and RR2 and the reflective surfaces RL1 and RL2 in the light guiding member IS1 will be described. A first method includes, as illustrated in FIG. 3, providing a plurality of wedge-shaped notches in a surface of a transparent material forming the light guiding member IS1 to form prisms between adjacent notches. Then, surfaces of the notches (prisms) are used as the refractive surfaces RR1 and RR2 or the reflective surfaces RL1 and RL2 (see FIG. 2 of Japanese Unexamined Patent Application Publication No. 2005-259361).

A second method includes radiating or focusing pulsed laser light on a transparent material forming the light guiding member IS1 to form regions with different refractive indices inside the transparent material. Then, the thus formed regions with different refractive indices are used as the refractive surfaces RR1 and RR2 or the reflective surfaces RL1 and RL2 (see Japanese Unexamined Patent Application Publication No. 2006-184890). The first and second methods can be applied in the same way to all the light guiding members used in each of the embodiments described below.

In the case where the optical measurement apparatus according to the first embodiment is provided in a cellular phone, for example, the light guiding member IS1 may be configured to serve also as an illumination of the cellular phone. In this case, of the elements illustrated in FIG. 3, the light source section LS1, the light receiving section LR1, and the light guiding member IS1 of the optical measurement apparatus are provided inside a cellular phone 100. Then, in the case where the optical measurement apparatus is not used to measure the thickness of the fat layer L2 or the like, the light guiding member IS1 functions as an illumination by emitting the lights B1 and B2 out of the cellular phone 100 through the emission positions 11 and 12, respectively.

In this case, the light source section LS1 is mounted on a wiring substrate P1 provided inside the cellular phone 100 through a surface opposite the light emitting surface LES1. Likewise, the light receiving section LR1 is mounted on a wiring substrate P2 provided inside the cellular phone 100 through a surface opposite the light receiving surface LRS1. In addition, the light guiding member IS1 is mounted on a wiring substrate P3 provided inside the cellular phone 100. While the lower surfaces of the light guiding member IS1 and the light receiving section LR1 in FIG. 3 are exposed to be in direct contact with the epidermis and dermis (L1) of the living body to be measured, the other portions are covered by an outer wall W1 of the cellular phone 100. In the case where the optical measurement apparatus is provided in the cellular phone in this way, the cellular phone itself may be deemed as an optical measurement apparatus at least in a scene where the cellular phone is used as an optical measurement apparatus. This also applies to each of the embodiments discussed below.

As described above with reference to FIGS. 1 and 2, the length L11 of the LED serving as the light source section LS1 in the direction orthogonal to the light emitting surface LES1 is longer than the length L12 of the LED along the light emitting surface LES1 (L11>L12). In the optical measurement apparatus according to the first embodiment, as illustrated in FIG. 3, the light source section LS1 is disposed with the light emitting surface LES1 of the light source section LS1 facing a direction orthogonal to the light receiving surface LRS1 of the light receiving section LR1 (directed rightward in FIG. 3), and the light receiving surface LRS1 of the light receiving section LR1 faces the epidermis and dermis (L1) of the living body to be measured. That is, the longitudinal direction of the light source section LS1 coincides with the direction of extension of the living body to be measured, and in the case where the optical measurement apparatus is provided in the cellular phone 100, also coincides with the direction of extension of an outer surface of the cellular phone 100 at the same time. In the arrangement of FIG. 3 (FIG. 2), the height of the light source section LS1 along the vertical direction in FIG. 3 is smaller compared to that in the arrangement of FIG. 1. As a result, in the optical measurement apparatus according to the first embodiment, the sensor section can be reduced in thickness at portions corresponding to the light source section LS1 and the light guiding member IS1. In the case where the optical measurement apparatus is provided in the cellular phone 100, a reduction in thickness of the portions corresponding to the light source section LS1 and the light guiding member IS1 improves the degree of freedom in the design of the cellular phone 100, and contributes to a reduction in thickness of the cellular phone 100.

According to the optical measurement apparatus of the first embodiment illustrated in FIG. 3, the positions and the number of the refractive surfaces RR1 and RR2 and the reflective surfaces RL1 and RL2 provided in the light guiding member IS1 can be selected as desired. As a result, a desired number of artificial light sources can be provided at desired positions.

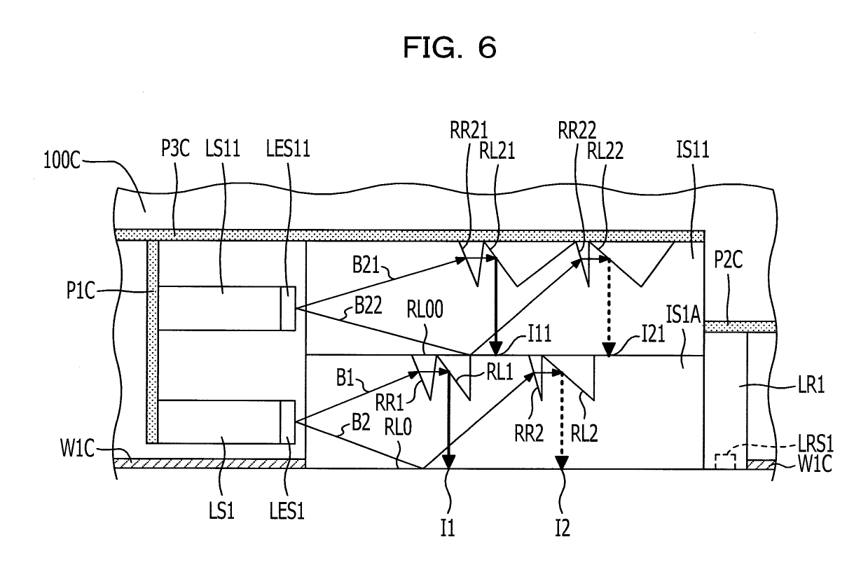

By adopting a configuration in which a plurality of light guiding members are laminated on each other as in a fourth embodiment to be discussed later with reference to FIG. 6, a plurality of light source sections can be disposed at a narrow pitch in correspondence with the plurality of light guiding members. As a result, the light guiding members can be used also as illuminations as described above. By providing the plurality of light source sections with different wavelengths from each other, it is possible to provide the optical measurement apparatus with a function to also acquire information on a substance in the living body other than the subcutaneous fat by utilizing the characteristics that different substances in the living body are different in light absorption properties as described above.

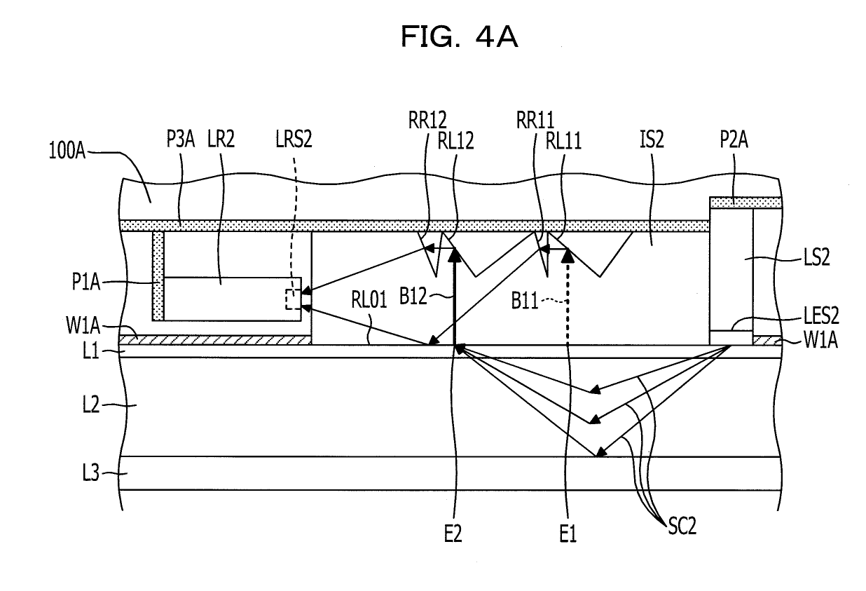

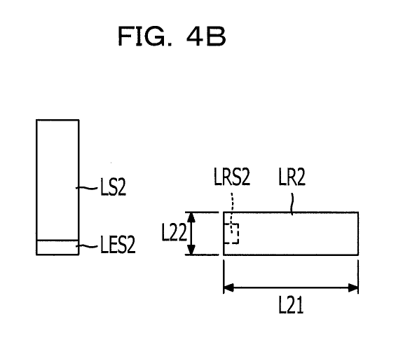

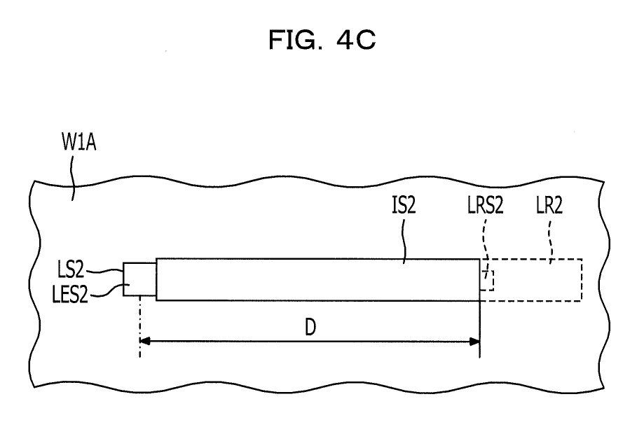

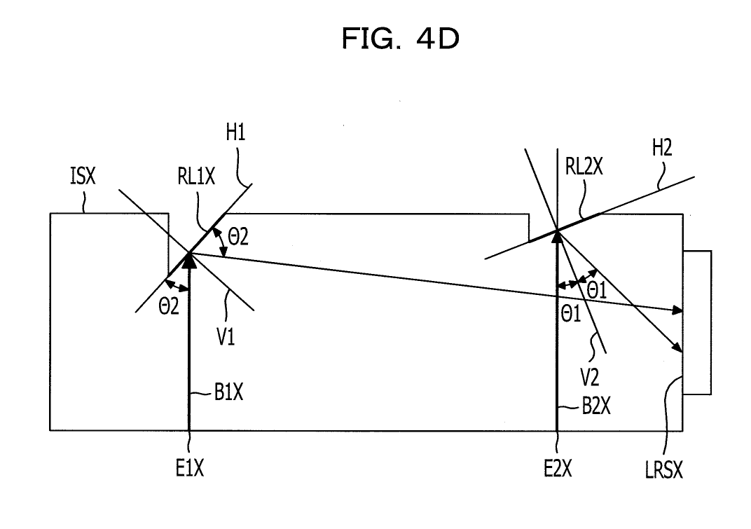

Next, an optical measurement apparatus according to a second embodiment will be described with reference to FIGS. 4A, 4B, 4C, and 4D.

The optical measurement apparatus according to the second embodiment uses the same principle as that of the optical measurement apparatus according to the first embodiment discussed above. Also in the second embodiment, as in the first embodiment, as illustrated in FIGS. 4A and 4B, a light source section LS2 is disposed with a light emitting surface LES2 of the light source section LS2 facing a direction orthogonal to a light receiving surface LRS2 of a light receiving section LR2 (downward in FIGS. 4A and 4B). Unlike in the first embodiment, however, the light emitting surface LES2 of the light source section LS2 faces the epidermis and dermis (L1) of the living body to be measured. That is, in the second embodiment, the longitudinal direction of the light receiving section LR2 coincides with the direction of extension of the living body to be measured, and in the case where the optical measurement apparatus is provided in a cellular phone 100A, also coincides with the direction of extension of an outer surface of the cellular phone 100A at the same time. As illustrated in FIG. 4B, a length L21 of the light receiving section LR2 in the direction orthogonal to the light receiving surface LRS2 is longer than a length L22 of the light receiving section LR2 along the light receiving surface LRS2 (L21>L22). As a result, in the arrangement of FIGS. 4A and 4B, the height of the light receiving section LR2 along the vertical direction in FIGS. 4A and 4B is smaller compared to the arrangement of FIG. 1. As a result, in the optical measurement apparatus according to the second embodiment, a sensor section can be reduced in thickness at portions corresponding to the light receiving section LR2 and a light guiding member IS2. In the case where the optical measurement apparatus is provided in the cellular phone 100A, a reduction in thickness of the portions corresponding to the light receiving section LR2 and the light guiding member IS2 improves the degree of freedom in the design of the cellular phone 100A, and contributes to a reduction in thickness of the cellular phone 100A.

The light source section LS2 and the light receiving section LR2 in the optical measurement apparatus according to the second embodiment may have the same configuration as that of the light source section LS1 and the light receiving section LR1, respectively, in the optical measurement apparatus according to the first embodiment described above.

In the optical measurement apparatus according to the second embodiment illustrated in FIGS. 4A and 4B, light emitted from the light emitting surface LES2 of the light source section LS2 is directly incident into the living body to be measured (L1→L2). After that, as in the first embodiment, the incident light is scattered in the fat layer L2 to be absorbed by the muscle layer L3, or is reflected by the muscle layer L3. Part of the light scattered in the fat layer L2 or reflected by the muscle layer L3, that is, SC2, is emitted out of the living body through the surface of the living body to be incident into the light guiding member IS2 through a lower surface of the light guiding member IS2 which is in contact with the surface of the living body in FIG. 4A. The light is emitted from the living body to travel in a direction substantially perpendicular (normal) to an outer surface of the epidermis and dermis L1 of the living body.

Of propagation light incident into the light guiding member IS2 in this way, light B11 incident at an emission position E1 travels upward and thereafter is reflected by a reflective surface RL11 provided in the light guiding member IS2 to travel substantially horizontally leftward as illustrated in FIG. 4A. After that, the light B11 is refracted by a refractive surface RR11 provided in the light guiding member IS2 to travel obliquely leftward and downward, is next reflected by a reflective surface RL01 provided in the light guiding member IS2 to travel obliquely leftward and upward, and reaches the light receiving surface LRS2 of the light receiving section LR2 to be received.

Likewise, of the light incident into the light guiding member IS2, light B12 incident at an emission position E2 travels upward and thereafter is reflected by a reflective surface RL12 provided in the light guiding member IS2 to travel substantially horizontally leftward as illustrated in FIG. 4A. After that, the light B12 is refracted by a refractive surface RR12 provided in the light guiding member IS2 to travel obliquely leftward and downward, and reaches the light receiving surface LRS2 of the light receiving section LR2 to be received.

The light guiding member IS2 in the optical measurement apparatus according to the second embodiment illustrated in FIG. 4A can be manufactured in the same method and using the same material as those for the light guiding member IS1 in the optical measurement apparatus according to the first embodiment discussed above. Also, the reflective surfaces RL11 and RL12 and the refractive surfaces RR11 and RR12 of the light guiding member IS2 can be formed in the same method (the first method or the second method) as that for the reflective surfaces RL1 and RL2 and the refractive surfaces RR1 and RR2 of the light guiding member IS1.

In the case where the optical measurement apparatus according to the second embodiment is provided in the cellular phone 100A, of the elements illustrated in FIG. 4A, the light source section LS2, the light receiving section LR2, and the light guiding member IS2 of the optical measurement apparatus are provided inside the cellular phone 100A. In this case, the light receiving section LR2 is mounted on a wiring substrate P1A provided inside the cellular phone 100A through a surface opposite the light receiving surface LRS2. Likewise, the light source section LS2 is mounted on a wiring substrate P2A provided inside the cellular phone 100A through a surface opposite the light emitting surface LES2. In addition, the light guiding member IS2 is mounted on a wiring substrate P3A provided inside the cellular phone 100A. While the lower surfaces of the light guiding member IS2 and the light source section LS2 in FIG. 4A are exposed to be in direct contact with the epidermis and dermis (L1) of the living body to be measured, the other portions are covered by an outer wall W1A of the cellular phone 100A.

FIG. 4C is a bottom view of the light source section LS2, the light receiving section LR2, and the light guiding member IS2 included in the sensor section of the optical measurement apparatus according to the second embodiment discussed above with reference to FIGS. 4A and 4B. In FIG. 4C, light emitted from the light emitting surface LES2 of the light source section LS2 toward the living body is incident into the living body (not illustrated in FIG. 4C). Part of the light scattered in the fat layer L2 or reflected by the muscle layer L3 in the living body is emitted through the surface of the living body to be incident into the light guiding member IS2. The light incident into the light guiding member IS2 is reflected by the light emitting surfaces RL11 and RL12 and refracted by the refractive surfaces RR11 and RR12 so as to be changed in traveling direction as discussed above with reference to FIG. 4A, and reaches the light receiving surface LRS2 of the light receiving section LR2 to be received. In FIG. 4C, the light emitting surface LES2 of the light source section LS2 may be square in shape and in 2 mm in height and width, for example. The distance D from the center of the light source section LS2 to the light receiving section LR2 may be 20 mm, for example. The cross-sectional area of the light guiding member IS2 taken along a plane in parallel with the light receiving surface LRS2 of the light receiving section LR2 (a plane orthogonal to the light emitting surface of FIG. 4C) is desirably four times or more the area of the light receiving surface LRS2 of the light receiving section LR2.

FIG. 4D illustrates the basic principle of the light guiding member IS2 provided in the optical measurement apparatus according to the second embodiment discussed above with reference to FIGS. 4A to 4C. For convenience of description, a side view of a light guiding member ISX having a different configuration from that of the light guiding member IS2 is used for the description. In FIG. 4D, part of the light scattered in the fat layer or reduced through absorption by the muscle layer in the living body (not illustrated in FIG. 4D) is emitted through the surface of the living body to be incident into the light guiding member ISX at emission positions E1X and E2X. Lights B1X and B2X traveling upward to be incident into the light guiding member ISX are reflected by reflective surfaces RL1X and RL2X, respectively, provided in the light guiding member ISX so as to travel obliquely rightward and downward in FIG. 4D, and reach a light receiving surface LRSX of the light receiving section to be received.

When the light B1X is reflected by the reflective surface RL1X, the relationship between a plane H1 extending along the reflective surface RL1X and a normal line V1 extending normal to the plane H1 and the traveling direction of the light B1X is as follows. That is, the angle θ2 formed between the light B1X incident on the reflective surface RL1X and the plane H1 is equal to the angle θ2 formed between the light B1X reflected by the reflective surface RL1X and the plane H1. This relationship is the same for the angles formed between the normal line V1 and the light B1X.

When the light B2X is reflected by the reflective surface RL2X, the relationship between a plane H2 extending along the reflective surface RL2X and a normal line V2 extending normal to the plane H2 and the traveling direction of the light B2X is as follows. That is, the angle θ1 formed between the light B2X incident on the reflective surface RL2X and the normal line V2 is equal to the angle θ1 formed between the light B2X reflected by the reflective surface RL2X and the normal line V2. This relationship is the same for the angles formed between the plane H2 and the light B2X.

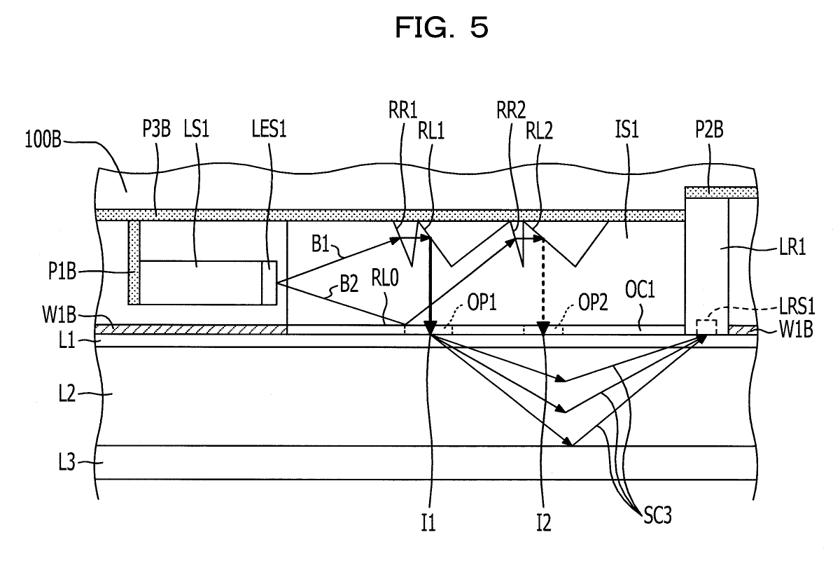

Next, the configuration of a light source section, a light receiving section, and a light guiding member included in a sensor section of an optical measurement apparatus according to a third embodiment will be described with reference to FIG. 5. The optical measurement apparatus according to the third embodiment of FIG. 5 has a similar configuration to that of the optical measurement apparatus according to the first embodiment discussed above with reference to FIG. 3. Thus, like constituent elements are denoted by like reference symbols to omit overlapping descriptions.

The optical measurement apparatus (third embodiment) of FIG. 5 is different from the optical measurement apparatus (first embodiment) of FIG. 3 in being provided with a light shielding member OC1. As illustrated in FIG. 5, the light shielding member OC1 is attached (for example, affixed using various types of bonding members such as an optical adhesive or an optical baseless double-sided adhesive tape) to cover a lower surface of the light guiding member IS1, that is, a surface of the light guiding member IS1 that is to face the epidermis and dermis (L1) of the living body to be measured. The light shielding member OC1 functions to prevent the lights B1, B2, and so forth emitted from the light source section LS1 and changed in traveling direction in the light guiding member IS1 from being incident into the living body to be measured (L1→L2) at positions other than the predetermined incident positions 11 and 12. In order to achieve the above function, the light shielding member OC1 has the shape of a plate that covers the lower surface of the light guiding member IS1, and is provided with opening portions OP1 and OP2 at portions corresponding to the predetermined incident positions I1 and I2, respectively.

The opening portions OP1 and OP2 of the light shielding member OC1 are configured to transmit the light emitted by the light source section LS1, that is, configured to be open, for example. The plate-shaped portion of the light shielding member OC1 other than the opening portions OP1 and OP2 is formed from a material that does not transmit (blocks) the light. The plate-shaped portion of the light shielding member OC1 other than the opening portions OP1 and OP2 can be manufactured by applying painting or metal plating to a surface of a silicon or ABS (Acrylonitrile Butadiene Styrene) plate material, for example. Each of the opening portions OP1 and OP2 may be a circular opening having a diameter of 2 mm or a square opening having a size of 2 mm by 2 mm.

As in each of the embodiments described above, the light source section LS1 is mounted on a wiring substrate P1B provided inside a cellular phone 100B through a surface opposite the light emitting surface LES1. Likewise, the light receiving section LR1 is mounted on a wiring substrate P2B provided inside the cellular phone 100B through a surface opposite the light receiving surface LRS1. In addition, the light guiding member IS1 is mounted on a wiring substrate P3B provided inside the cellular phone 100B. While the lower surfaces of the light shielding member OC1 and the light receiving section LR1 in FIG. 5 are exposed to be in direct contact with the epidermis and dermis (L1) of the living body to be measured, the other portions are covered by an outer wall W1B of the cellular phone 100B.

According to the optical measurement apparatus of the third embodiment of FIG. 5 discussed above, providing the light shielding member OC1 can prevent the light emitted from the light source section LS1 from being incident into the living body to be measured via the light guiding member IS1 at positions other than the predetermined incident positions. As a result, even in the case where the light guiding member IS1 has a scratch so that unintended light may be incident into the living body to be measured at positions other than the predetermined incident positions because of the presence of the scratch, for example, it is possible to prevent such unintended light from being incident into the living body. Hence, the measurement precision of the optical measurement apparatus can be improved.

In addition, according to the optical measurement apparatus of the third embodiment of FIG. 5, as in the first embodiment, the light guiding member IS1 can be used also as an illumination as described above.

Next, an optical measurement apparatus according to a fourth embodiment will be described with reference to FIG. 6. In the optical measurement apparatus according to the fourth embodiment, as illustrated in FIG. 6, two light guiding members are provided to overlap each other vertically, and respective light source sections are provided for the two light guiding members. Each of light guiding members IS1A and IS11 has the same configuration and function as those of the light guiding member IS1 according to the first embodiment discussed above with reference to FIG. 3, and changes the traveling direction of light emitted from the light emitting surfaces LES1 and LES11 of the corresponding light source sections LS1 and LS11, respectively. In this way, the light guiding members IS1A and IS11 change the traveling direction of the light emitted from the light source sections LS1 and LS11, respectively, so as to travel downward in FIG. 6 to be incident into a living body to be measured (not illustrated) facing a lower surface of the light guiding member IS1A. Part of the light emitted from the light source sections LS1 and LS11 to be incident into the living body in this way is scattered in the fat layer or reflected by the muscle layer in the living body, is emitted from a surface of the living body, and reaches the light receiving surface LRS1 of the light receiving section LR1 to be received as in the first embodiment described above.

According to the optical measurement apparatus of the fourth embodiment of FIG. 6, as in the first embodiment, the light guiding members IS1A and IS11 can be used also as illuminations as described above. In addition, the light source sections LS1 and LS11 can be configured to emit light at different wavelengths from each other. As a result, it is possible to provide the optical measurement apparatus with a function to also acquire information on a substance in the living body to be measured other than the subcutaneous fat by utilizing the characteristics that different substances in the living body are different in light absorption properties for various wavelengths as described above.

In FIG. 6, lights B1 and B2 emitted from the light source section LS1 are changed in traveling direction by the refractive surface RR1, the reflective surface RL1, the reflective surface RL0, the refractive surface RR2, and the reflective surface RL2 provided in the light guiding member IS1A to be incident into the living body to be measured at the incident positions I1 and I2, respectively. Likewise, lights B21 and B22 emitted from the upper light source section LS11 are changed in traveling direction by a refractive surface RR21, a reflective surface RL21, a reflective surface RL00, a refractive surface RR22, and a reflective surface RL22 provided in the light guiding member IS11. After being changed in traveling direction, the lights B21 and B22 are incident into the lower light guiding member IS1A at incident positions 121 and 122 and then transmit through the light guiding member IS1A to be incident into the living body to be measured.

In the fourth embodiment of FIG. 6, the light source sections LS1 and LS11 are mounted on a wiring substrate P1C provided inside a cellular phone 100C through respective surfaces opposite the light emitting surfaces LES1 and LES11. Likewise, the light receiving section LR1 is mounted on a wiring substrate P2C provided inside the cellular phone 100C through a surface opposite the light receiving surface LRS1. In addition, the two light guiding members IS1A and IS11 are affixed to each other using an adhesive or the like to be mounted on a wiring substrate P3C provided inside the cellular phone 100C. While the lower surfaces of the light guiding member IS11 and the light receiving section LR1 in FIG. 6 are exposed to be in direct contact with the epidermis and dermis (L1, not illustrated in FIG. 6) of the living body to be measured, the other portions are covered by an outer wall W1C of the cellular phone 100C.

In the fourth embodiment of FIG. 6, the two light guiding members IS1A and IS11 are arranged adjacent to each other vertically (in the perpendicular direction). However, the present invention is not limited thereto, and two light guiding members corresponding to the two light guiding members IS1A and IS11 may be arranged adjacent to each other in the horizontal direction (a direction parallel to the outer wall surface of FIG. 6). Whether to arrange the two light guiding members in the perpendicular direction or in the horizontal direction may be determined in accordance with an installation space in the cellular phone or the like for installation of the optical measurement apparatus including the light guiding members, for example. Further, the number of the light guiding members to be arranged adjacently is not limited to two as in the example described above, and may be three or more. In such cases, three or more light source sections may be provided correspondingly.

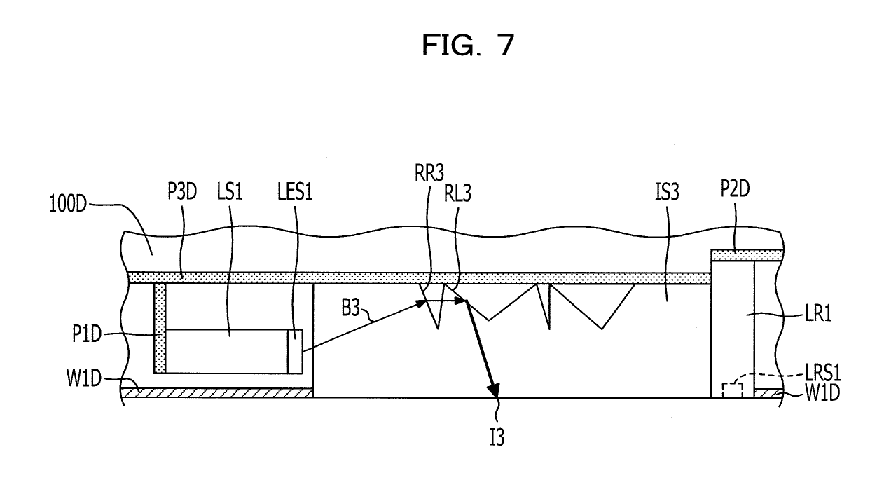

Next, an optical measurement apparatus according to a fifth embodiment will be described with reference to FIG. 7. The optical measurement apparatus according to the fifth embodiment has a similar configuration to that of the optical measurement apparatus according to the first embodiment discussed above with reference to FIG. 3. Thus, like constituent elements are denoted by like reference symbols to omit overlapping descriptions. A light guiding member IS3 used in the fifth embodiment can be manufactured in the same method and using the same material as those for the light guiding member IS1 used in the first embodiment.

In the optical measurement apparatus of FIG. 7, a refractive surface RR3 and a reflective surface RL3 of the light guiding member IS3 are formed as follows. That is, the refractive surface RR3 and the reflective surface RL3 of the light guiding member IS3 are formed to change the traveling direction of light emitted from the light source section LS1 such that the light is incident into a living body to be measured (not illustrated in FIG. 7) with an inclination with respect to a surface of the living body as illustrated in FIG. 7. That is, when light B3 is incident into the living body at an incident position 13 after being changed in traveling direction by the light guiding member IS3, the light B3 is incident with an inclination with respect to the direction perpendicular to the surface of the living body. The direction of the inclination is set such that the light approaches the light receiving section LR1 toward the living body as illustrated in FIG. 7. That is, the light incident into the living body is inclined toward the light receiving section LR1 at a certain angle. This makes it easy for light scattered or reflected inside the living body to be measured to arrive at the light receiving section LR1 even in the case where an LED that emits a relatively small amount of light is used as the light source section LS1, for example.

In the configuration of FIG. 7, the light B3 emitted by the light source section SL1 is refracted by the refractive surface RR3 of the light guiding member IS3 to travel substantially horizontally rightward and thereafter reflected by the reflective surface RL3 of the light guiding member IS3 to travel obliquely rightward and downward to be incident into the living body to be measured at the incident position 13.

In the configuration of FIG. 7, the light source section LS1 is mounted on a wiring substrate P1D provided inside a cellular phone 100D through a surface opposite the light emitting surface LES1. Likewise, the light receiving section LR1 is mounted on a wiring substrate P2D provided inside the cellular phone 100D through a surface opposite the light receiving surface LRS1. In addition, the light guiding member IS3 is mounted on a wiring substrate P3D provided inside the cellular phone 100D. While the lower surfaces of the light guiding member IS3 and the light receiving section LR1 in FIG. 7 are exposed to be in direct contact with the epidermis and dermis (L1, not illustrated in FIG. 7) of the living body to be measured, the other portions are covered by an outer wall W1D of the cellular phone 100D.

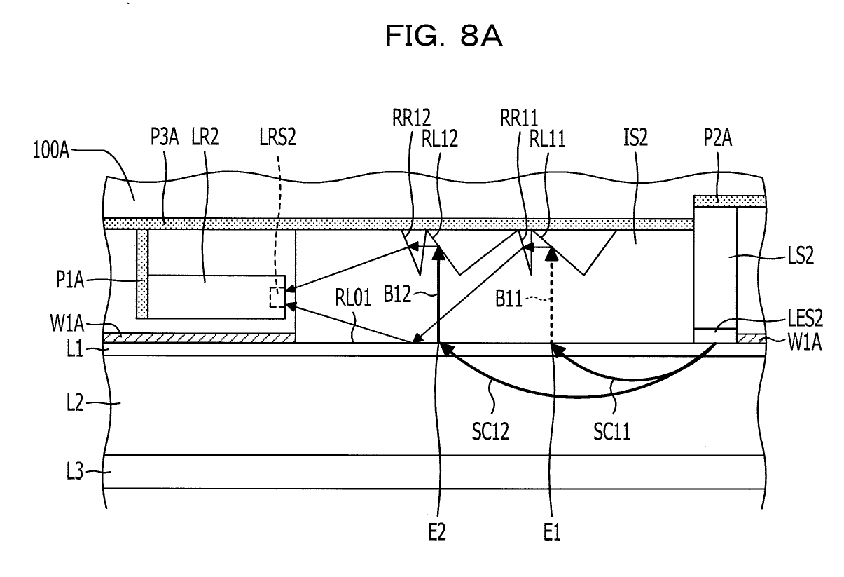

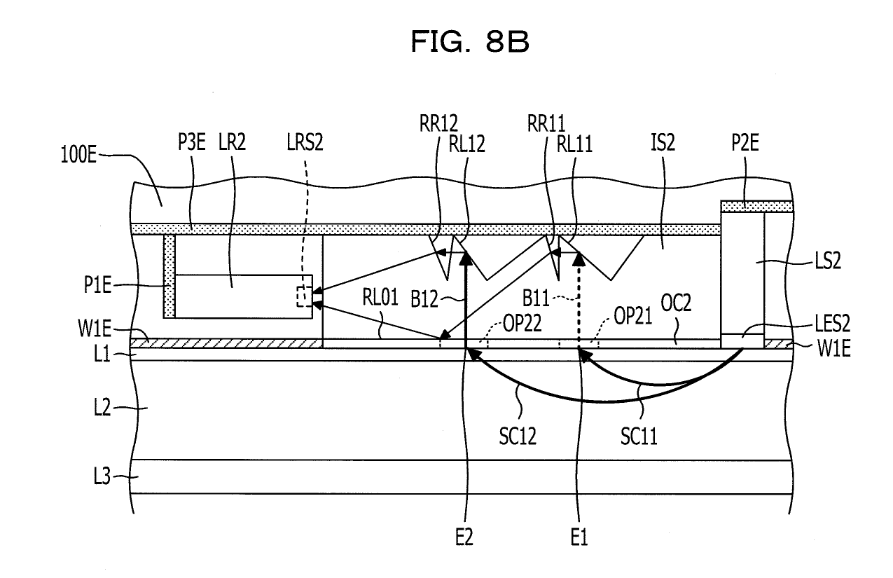

Next, an optical measurement apparatus according to a sixth embodiment will be described with reference to FIGS. 8A and 8B in comparison with the optical measurement apparatus according to the second embodiment discussed above.

FIG. 8A is a side view illustrating an example of the arrangement of the light source section LS2, the light receiving section LR2, and the light guiding member IS2 of the optical measurement apparatus according to the second embodiment discussed above with reference to FIG. 4A. In FIG. 8A, as discussed above with reference to FIG. 4A, light emitted from the light source section LS2 is incident into the living body to be measured (L1→L2), and scattered in the fat layer L2 or reflected by the muscle layer L3. Lights SC11 and SC12, which are each part of the incident light described above, are emitted from the living body at emission positions E1 and E2 (lights B11 and B12), respectively, and changed in traveling direction by the reflective surface RL11, the refractive surface RR11, the reflective surface RL01, the reflective surface RL12, and the refractive surface RR12. After being changed in traveling direction in this way, the lights B11 and B12 are guided to the light receiving surface LRS2 of the light receiving section LR2 to be received.

FIG. 8B is a side view illustrating an example of the arrangement of the light source section LS2, the light receiving section LR2, and the light guiding member IS2 of the optical measurement apparatus according to the sixth embodiment. The sixth embodiment of FIG. 8B is different from the second embodiment discussed above with reference to FIGS. 4A and 8A in having a light shielding member OC2 as with the third embodiment discussed above with reference to FIG. 5. The sixth embodiment is otherwise the same as the second embodiment. Thus, like constituent elements are denoted by like reference symbols to omit overlapping descriptions.

As illustrated in FIG. 8B, the light shielding member OC2 is attached (for example, affixed using an adhesive or the like) to cover a lower surface of the light guiding member IS2, that is, a surface of the light guiding member IS2 that is to face the epidermis and dermis (L1) of the living body to be measured. The light shielding member OC2 functions to prevent part of propagation light emitted from the light source section LS2 to be incident into the living body to be measured (L1→L2) from being emitted from the living body to be incident into the light guiding member IS3 at positions other than the predetermined emission positions E1 and E2. In order to achieve the above function, the light shielding member OC2 has the shape of a plate that covers the lower surface of the light guiding member IS2, and is provided with opening portions OP21 and OP22 at portions corresponding to the predetermined emission positions E1 and E2, respectively. The opening portions OP21 and OP22 of the light shielding member OC2 are configured to transmit the light emitted by the light source section LS2, that is, configured to be open, for example. The plate-shaped portion of the light shielding member OC2 other than the opening portions OP21 and OP22 is formed from a material that does not transmit (blocks) the light. The plate-shaped portion of the light shielding member OC2 other than the opening portions OP21 and OP22 can be manufactured by applying painting or metal plating to a surface of a silicon or ABS (Acrylonitrile Butadiene Styrene) plate material, for example. Each of the opening portions OP21 and OP22 may be a circular opening having a diameter of 2 mm or a square opening having a size of 2 mm by 2 mm.

As in each of the embodiments described above, the light source section LS2 is mounted on a wiring substrate P1E provided inside a cellular phone 100E through a surface opposite the light emitting surface LES2. Likewise, the light receiving section LR2 is mounted on a wiring substrate P2E provided inside the cellular phone 100E through a surface opposite the light receiving surface LRS2. In addition, the light guiding member IS2 is mounted on a wiring substrate P3E provided inside the cellular phone 100E. While the lower surfaces of the light guiding member IS2 and the light source section LS2 in FIG. 8B are exposed to be in direct contact with the epidermis and dermis (L1) of the living body to be measured, the other portions are covered by an outer wall W1E of the cellular phone 100E.

According to the optical measurement apparatus of the sixth embodiment discussed above with reference to FIG. 8B, providing the light shielding member OC2 can prevent return light, of the light emitted from the light source section LS2 to be incident into the living body to be measured, from being emitted from the living body to be incident into the light guiding member IS2 at positions other than the predetermined emission positions. As a result, even in the case where the light guiding member IS2 has a scratch so that unintended light may be incident into the light guiding member IS2 at positions other than the predetermined emission positions E1 and E2 because of the presence of the scratch to be guided to the light receiving section LR2, for example, it is possible to prevent such unintended light from being guided to the light receiving section LR2. Hence, the measurement precision of the optical measurement apparatus can be improved.

Next, a method of improving the precision of the optical measurement apparatus according to each of the embodiments described above in measuring the thickness of subcutaneous fat will be described with reference to FIG. 9. In FIG. 9, the horizontal axis represents the horizontal distance (mm) over which propagation light propagates through the living body to be measured, and the vertical axis represents a voltage value (output voltage (V)) of a signal output by the light receiving section. The values indicated in FIG. 9 are merely examples for illustration, and none of the embodiments are limited by such values. As illustrated in FIG. 9, the output voltage for the same horizontal distance is different between thicknesses (T=8 (mm), T=12 (mm), and T=40 (mm)) of the fat layer L2 to be measured. More specifically, the output voltage for a certain horizontal distance is higher as the fat layer L2 is thicker. Hence, the thickness of the fat layer L2 can be obtained from the output voltage.

In each of the embodiments described above, light is incident into the living body to be measured at the plurality of incident positions I1 and I2, or emitted from the living body to be measured at the plurality of emission positions E1 and E2, to be used in measurement as described above. That is, light is incident into the living body to be measured at the plurality of incident positions I1 and I2 which are different from each other, or emitted from the living body to be measured at the plurality of emission positions E1 and E2 which are different from each other, for use in measurement. As a result, it is possible to obtain from the light receiving section an output signal indicating a value obtained by totaling a plurality of sets of measurement results for different horizontal distances over which propagation light propagates through the living body to be measured.

That is, in the configuration of FIG. 3, for example, the horizontal distance over which light incident into the living body to be measured L1 to L3 at the incident position I1 propagates in the living body before reaching the light receiving surface LRS1 of the light receiving section LR1 is assumed to be 30 mm. Likewise, the horizontal distance over which light incident into the living body to be measured at the incident position 12 propagates in the living body before reaching the light receiving surface LRS1 of the light receiving section LR1 is assumed to be 20 mm. In this case, the respective lights incident at the incident positions I1 and I2 reach the light receiving surface LRS1 of the light receiving section LR1 to be received. The amount of the light received at that time is the total of the respective amounts of the received lights resulting from the respective lights incident at the incident positions I1 and I2.

In FIG. 9, for example, the thickness of the fat layer L2 to be measured is assumed to be T=40 (mm). Then, the output voltage is generally 0.3 V in the case where the horizontal propagation distance is 30 mm as described above, and generally 1.2 V in the case where the horizontal propagation distance is 20 mm. Thus, in this case, the total amount of the received light is indicated by an output voltage of generally 1.5 V (0.3+1.2=1.5). Likewise, the thickness of the fat layer L2 is assumed to be T=12 (mm). Then, the output voltage is generally 0.2 V in the case where the horizontal propagation distance is 30 mm as described above, and generally 0.8 V in the case where the horizontal propagation distance is 20 mm. Thus, in this case, the total amount of the received light is indicated by an output voltage of generally 1.0 V (0.2+0.8=1.0). As a result, the difference between the output voltages is generally 0.5 V (1.5−1.0=0.5), which is apparently enhanced compared to each of a difference of 0.1 V (0.3−0.2=0.1) obtained in the case where the horizontal propagation distance is 30 mm and a difference of 0.4 V (1.2−0.8=0.4) obtained in the case where the horizontal propagation distance is 20 mm.

In this way, in each of the embodiments, measurement is performed using the total of the respective amounts of the lights received after propagating over a plurality of different horizontal distances. Therefore, the difference in output voltage can be enhanced with respect to the difference in thickness of the fat layer L2, which improves the S/N (Signal/Noise) ratio for a diversity of the measured thickness of subcutaneous fat.

Next, an example of the overall functional configuration of the optical measurement apparatus according to each of the embodiments described above will be described with reference to FIGS. 10A and 10B.

The optical measurement apparatus according to each of the embodiments roughly includes a sensor section 10, a subcutaneous fat thickness calculation device 20, and an output section 31. The sensor section 10 includes a light emission control section 11, a light emitting section 12, and a light receiving section 13. The light emitting section 11 signifies each of the light source sections LS1, LS2, and LS11 described above. The light receiving section 13 signifies each of the light receiving sections LR1 and LR2 described above. As described in relation to each of the embodiments described above, the light emitting section 12 emits light to the living body to be measured L1 to L3, and the light receiving section 13 receives return light from the living body. The light emission control section 11 controls a value of a current to be supplied to the light emitting section 11 (for example, an LED) to control the amount of the light emitted by the light emitting section 11.

The subcutaneous fat thickness calculation device 20 includes a data storage section 21, a data reliability determination section 22, a subcutaneous fat thickness calculation section 23, and a subcutaneous fat thickness determination section 24. The data storage section 21 stores a voltage value of an output signal output from the light receiving section 13 corresponding to the amount of the received light.

The data reliability determination section 22 controls the light emission control section 11 so as to create a state in which the light emitting section 12 emits no light in a first step. In the state in which the light emitting section 12 emits no light, the data storage section 21 stores the voltage value of the output signal indicating the amount of the light received by the light receiving section 13. The data reliability determination section 22 reads the stored voltage value from the data storage section 21 to determine whether or not the read voltage value is equal to or less than a predetermined threshold. By performing this determination, the data reliability determination section 22 confirms whether or not the light receiving surface of the light receiving section 13 is in contact with the surface of the living body to be measured L1 to L3. If the light receiving surface of the light receiving section 13 is not in contact with the surface of the living body, the light receiving section 13 receives light such as sunlight, illumination light from a room lighting fixture, or the like, and the amount of received light is obtained as if the light emitting section 12 is emitting light. As a result, the voltage value of the output signal from the light receiving section 13 exceeds the threshold, which allows detection of a state in which the light receiving surface of the light receiving section 13 is not in contact with the surface of the living body. Thus, in the case where the light receiving surface of the light receiving section 13 is not in contact with the surface of the living body, accurate measurement may not be performed so that an error output is issued to the outside via the output section 31, for example.

On the other hand, in the case where the stored voltage value read from the data storage section 21 by the data reliability determination section 22 is equal to or less than the predetermined threshold in the first step, a second step is executed next. In the second step, the data reliability determination section 22 controls the light emission control section 11 so as to increase the amount of the light emitted by the light emitting section 12 stepwise at a constant rate of increase (stepwise light emitting operation). As a result, voltage values of the output signal from the light receiving section 13, which indicate the respective light reception amounts corresponding to the respective light emission amounts of the light emitting section 12 increased stepwise, are stored in the data storage section 21.

Next, in a third step, the data reliability determination section 22 controls the light emission control section 11 so as to create a state in which the light emitting section 12 emits no light as in the first step. The data reliability determination section 22 confirms whether or not the light receiving surface of the light receiving section 13 is in contact with the surface of the living body to be measured in the same way as in the first step. Also in this case, as in the first step, in the case where the stored voltage value read from the data storage section 21 is equal to or less than the predetermined threshold, the data reliability determination section 22 issues an error output to the outside via the output section 31, for example.

In the case where the stored voltage value read from the data storage section 21 is equal to or less than the predetermined threshold in the third step, the data reliability determination section 22 executes a fourth step. In the fourth step, the data reliability determination section 22 reads a voltage value indicating a light reception amount corresponding to a predetermined light emission amount, from the voltage values indicating the respective light reception amounts corresponding to the respective light emission amounts stored in the data storage section 21 in the stepwise light emitting operation performed in the second step, to output the read voltage value to the subcutaneous fat thickness calculation section 23.

The subcutaneous fat thickness calculation section 23 calculates the thickness of the fat layer L2 (subcutaneous fat thickness) of the living body to be measured on the basis of the voltage value output from the data reliability determination section 22 by a method to be discussed later with reference to FIG. 10B. The subcutaneous fat thickness determination section 24 determines whether or not a value of the thickness of the fat layer L2 calculated by the subcutaneous fat thickness calculation section 23 falls within a range predicted in advance. If the value of the calculated thickness of the fat layer L2 does not fall within the range predicted in advance, the subcutaneous fat thickness determination section 24 issues an error output to the output section 31. If the value of the calculated thickness of the fat layer L2 falls within the range predicted in advance, the subcutaneous fat thickness determination section 24 outputs a value of the calculated thickness of the fat layer L2 to the output section 31. The output section 31 may be a display device such as an LCD (Liquid Crystal Display), for example, and displays the value output from the subcutaneous fat thickness determination section 24, or issues an error output as described above.

Next, the method of calculating the thickness of the fat layer L2 performed by the subcutaneous fat thickness calculation section 23 will be described with reference to FIG. 10B. FIG. 10B is a graph (curve) representing the relationship between the voltage value (light reception amount [V]) of the output signal indicating the light reception amount of the light receiving section 13 corresponding to the predetermined light emission amount of the light emitting section 12 and the thickness (calculated fat thickness [mm]) of the fat layer L2 of the living body to be measured. As illustrated in FIG. 10B, the thickness of the fat layer L2 of the living body to be measured is larger as the voltage value of the output signal indicating the light reception amount of the light receiving section 13 is larger. The graph of FIG. 10B may be represented by a function formula y=a×Ln(x)+b, for example. In the function formula, y represents the thickness (calculated fat thickness [mm]) of the fat layer L2 of the living body to be measured, and x represents the voltage value (light reception amount [V]) of the output signal indicating the light reception amount of the light receiving section 13. Thus, the subcutaneous fat thickness calculation section 23 can obtain the thickness (calculated fat thickness [mm]) of the fat layer L2 of the living body to be measured from the voltage value (light reception amount [V]) of the output signal indicating the light reception amount of the light receiving section 13 corresponding to the predetermined light emission amount, which is output from the data reliability determination section 22, using the graph of FIG. 10B or the function formula.

According to an aspect of the embodiments of the invention, any combinations of one or more of the described features, functions, operations, and/or benefits can be provided. A combination can be one or a plurality. The embodiments can be implemented as an apparatus (a machine) that includes hardware for performing the described features, functions, operations, and/or benefits, for example, optical hardware (e.g., light source, etc.), hardware to execute instructions, for example, computing hardware (i.e., computing apparatus), such as (in a non-limiting example) any computer that can store, retrieve, process and/or output data and/or communicate (network) with other computers. According to an aspect of an embodiment, the described features, functions, operations, and/or benefits can be implemented by and/or use computing hardware and/or software. An optical measurement device, such as the optical measurement device 20, can comprise a controller (CPU) (e.g., a hardware logic circuitry based computer processor that processes or executes instructions, namely software/program), computer readable media, transmission communication interface (network interface), and/or an output device, for example, a display device, and which can be in communication among each other through one or more data communication buses. In addition, an apparatus can include one or more apparatuses in computer network communication with each other or other apparatuses. In addition, a computer processor can include one or more computer processors in one or more apparatuses or any combinations of one or more computer processors and/or apparatuses. An aspect of an embodiment relates to causing one or more apparatuses and/or computer processors to execute the described operations. The results produced can be output to an output device, for example, displayed on the display. An apparatus or device refers to a physical machine, for example, a computer (physical computing hardware or machinery) that implement or execute instructions, for example, by way of software, which is code executed by computing hardware, and/or by way of computing hardware (e.g., in circuitry, etc.), to achieve the functions or operations being described. The functions of embodiments described can be implemented in any type of apparatus that can execute instructions or code. More particularly, programming or configuring or causing an apparatus or device, for example, a computer, to execute the described functions of embodiments of the invention creates a new machine where in case of a computer a general purpose computer in effect becomes a special purpose computer once it is programmed or configured or caused to perform particular functions of the embodiments of the invention pursuant to instructions from program software.

A program/software implementing the embodiments may be recorded on a computer-readable media, e.g., a non-transitory or persistent computer-readable medium. Examples of the non-transitory computer-readable media include a magnetic recording apparatus, an optical disk, a magneto-optical disk, and/or volatile and/or non-volatile semiconductor memory (for example, RAM, ROM, etc.). Examples of the magnetic recording apparatus include a hard disk device (HDD), a flexible disk (FD), and a magnetic tape (MT). Examples of the optical disk include a DVD (Digital Versatile Disc), DVD-ROM, DVD-RAM (DVD-Random Access Memory), BD (Blue-ray Disk), a CD-ROM (Compact Disc-Read Only Memory), and a CD-R (Recordable)/RW. The program/software implementing the embodiments may be transmitted over a transmission communication path, e.g., a wire and/or a wireless network implemented via hardware. An example of communication media via which the program/software may be sent includes, for example, a carrier-wave signal.

According to an aspect of an embodiment, the sensor section 10 and one or more sections of an optical measurement device, such as the optical measurement device 20, are provided for (e.g., inside) a mobile device, such as a cellular phone or other wireless communication device, and the described functions are configured and/or controlled locally through the mobile device user interface and/or caused to be configured and/or controlled remotely via wire and/or wireless data communication (e.g., cellular network including Internet). According to an aspect of an embodiment, output signals from the sensor section 10 is directly and/or indirectly provided to another device that provide optical measurement functionality via wire and/or wireless data communication (e.g., cellular network including Internet) for optical measurement calculation based upon the signals output from the sensor section 10 and to further output (e.g., display) at the other (e.g., remote) device.

All examples and conditional language recited herein are intended for pedagogical objects to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment(s) of the present inventions have been described in detail, it should be understood that the various changes, substitutions, and alterations could be made hereto without departing from the spirit and scope of the invention.

What is claimed is:

1. An optical measurement apparatus for a body, comprising:
a light source section having a light emitting surface that emits light to be incident into the body;
a light receiving section having a light receiving surface that receives the light which is emitted by the light emitting surface of the light source section to be incident into the body through a surface of the body and which is emitted out of the body after propagating inside the body, the light receiving surface facing a direction orthogonal to the light emitting surface of the light source section, and the light receiving section configured to output a signal having a value corresponding to an amount of the light received by the light receiving surface; and
a light guiding section provided on a path of the light emitted from the light emitting surface of the light source section and received by the light receiving surface of the light receiving section to change a traveling direction of the light.

2. The optical measurement apparatus according to claim 1,
wherein the light guiding section includes a first light guiding member that changes the traveling direction of the light emitted out of the body after propagating through the body to guide the light to the light receiving surface of the light receiving section.

3. The optical measurement apparatus according to claim 1,
wherein the light guiding section includes a light guiding member that changes the traveling direction of the light which is emitted by the light emitting surface of the light source section to guide the light into the body through the surface of the body.

4. The optical measurement apparatus according to claim 2,
wherein the light guiding section includes a second light guiding member that changes the traveling direction of the light which is emitted by the light emitting surface of the light source section to guide the light into the body through the surface of the body.

5. The optical measurement apparatus according to any one of claim 1,
wherein the light guiding section includes one or more of a reflective surface that reflects the light to change the traveling direction of the light and a refractive surface that refracts the light to change the traveling direction of the light.

6. The optical measurement apparatus according to claim 3,
wherein the light guiding section changes the traveling direction of the light which is emitted by the light emitting surface of the light source section such that the light is incident into the body through the surface of the body at a plurality of incident positions.

7. The optical measurement apparatus according to claim 2,
wherein the light guiding section changes the traveling direction of the light, which is emitted by the light emitting surface of the light source section to be incident into the body through the surface of the body and which is emitted out of the body through the surface of the body at a plurality of emission positions after propagating inside the body, to guide the light to the light receiving surface of the light receiving section.

8. The optical measurement apparatus according to any one of claim 1,
wherein the light source section includes a plurality of light source sections with different wavelengths,
the light guiding section includes a plurality of light guiding members with respective reflective and/or refractive surfaces respectively provided for the plurality of light source sections, and
light emitted by a reflective and/or refractive surface for one of the plurality of light source sections is changed in traveling direction by one of the plurality of light guiding members provided for the one light source section to be incident into the body through the surface of the body, and the light which is emitted out of the body through the surface of the living body after propagating inside the body is received by the light receiving surface of the light receiving section.

9. A mobile wireless communication device, comprising:
a sensor section that includes:
a light source section having a light emitting surface that emits light to be incident into a body,
a light receiving section having a light receiving surface that receives the light which is emitted by the light emitting surface of the light source section to be incident into the body through a surface of the body and which is emitted out of the body after propagating inside the body, the light receiving surface facing a direction orthogonal to the light emitting surface of the light source section, and the light receiving section is configured to output a signal having a value corresponding to an amount of the light received by the light receiving surface, and
a light guiding section provided on a path of the light emitted from the light emitting surface of the light source section and received by the light receiving surface of the light receiving section to change a traveling direction of the light; and
a computer controller that obtains information of a measurement with respect to the body according to the signal output by the light receiving section.
